(19) United States
(12) Patent Application Publication
(10) Pub. No.: US 2002/0065228 A1
(43) Pub. Date: May 30, 2002

(54) Insecticidal peptidomimetics of trypsin modulating oostatic factor

(76) Inventors: Russell J. Linderman, Green Oaks, IL (US); Dov Borovsky, Vero Beach, FL (US)

Correspondence Address: MYERS BIGEL SIBLEY & SAJOVEC, PO BOX 37428, RALEIGH, NC, 27627, US

(21) Appl. No.: 09/727,236

(22) Filed: Nov. 30, 2000

(51) Int. Cl. A61K 38/00; A61K 31/535; A61K 31/445; A61K 31/40; A61K 31/165

(52) U.S. Cl. 514/18; 514/19; 514/237.8; 514/331; 514/428; 514/617

(57) ABSTRACT

Methods of controlling a pest such as an insect pest comprise administering to said pest a pesticidally effective amount of a non-peptide TMOF analog (also referred to as an “active compound” or “pesticidal compound” herein). Compositions and compounds useful for carrying out such methods are also disclosed.

### FIELD OF THE INVENTION

The present invention concerns methods, compounds and compositions useful for the control of insect pests.

### BACKGROUND OF THE INVENTION

Many blood-ingesting pests are known to feed on humans and animals, and many pests are vectors for pathogenic microorganisms which threaten human and animal health, including commercially important livestock, pets and other animals. Various species of mosquitoes, for example, transmit diseases caused by viruses, and many are vectors for disease-causing nematodes and protozoa. Mosquitoes of the genus Anopheles transmit Plasmodium, the protozoan which causes malaria, a devastating disease which results in approximately 1 million deaths annually. The mosquito species Aedes aegypti transmits an arbovirus that causes yellow fever in humans. Other arboviruses transmitted by Aedes species include the causative agents of dengue fever, eastern and western encephalitis, Venezuelan equine encephalitis, St. Louis encephalitis, chikungunya, oroponehe and bunyarnidera. The genus Culex, which includes the common house mosquito C. pipiens, is implicated in the transmission of various forms of encephalitis and filarial worms. The common house mosquito also transmits Wuchereria banuffi and Brugia malayi, which cause various forms of lymphatic filariasis, including elephantiasis. Trypanasomas cruzi, the causative agent of Chagas' disease, is transmitted by various species of blood-ingesting Triatominae bugs. The tsetse fly (Glossina spp.) transmits African trypanosomal diseases of humans and cattle. Many other diseases are transmitted by various blood-ingesting pest species. The order Diptera contains a large number of blood-ingesting and disease-bearing insects, including, for example, mosquitoes, black flies, no-see-ums (punkies), horse flies, deer flies and tsetse flies.

Various pesticides have been employed in efforts to control or eradicate populations of disease-bearing pests, such as disease-bearing blood-ingesting pests. For example, DDT, a chlorinated hydrocarbon, has been used in attempts to eradicate malaria-bearing mosquitoes throughout the world. Other examples of chlorinated hydrocarbons are BHC, lindane, chlorobenzilate, methoxychlor, and the cyclodienes (e.g., aldrin, dieldrin, chlordane, heptachlor, and endrin). The long-term stability of many of these pesticides and their tendency to bioaccumulate render them particularly dangerous to many non-pest organisms.

Another common class of pesticides is the organophosphates, which is perhaps the largest and most versatile class of pesticides. Organophosphates include, for example, parathion, Malathion, diazinon, naled, methyl parathion, and dichlorvos. Organophosphates are generally much more toxic than the chlorinated hydrocarbons. Their pesticidal effect results from their ability to inhibit the enzyme cholinesterase, an essential enzyme in the functioning of the insect nervous system. However, they also have toxic effects on many animals, including humans.

The carbamates, a relatively new group of pesticides, include such compounds as carbamyl, methomyl, and carbofuran. These compounds are rapidly detoxified and eliminated from animal tissues. Their toxicity is thought to involve a mechanism similar to the mechanism of the organophosphates; consequently, they exhibit similar shortcomings, including animal toxicity.

A major problem in pest control results from the capability of many species to develop pesticide resistance. Resistance results from the selection of naturally-occurring mutants possessing biochemical, physiological or behavioristic factors that enable the pests to tolerate the pesticide. Species of Anopheles mosquitoes, for example, have been known to develop resistance to DDT and dieldrin. DDT substitutes, such as Malathion™, propoxur and fenitrothion are available; however, the cost of these substitutes is much greater than the cost of DDT.

There is clearly a longstanding need in the art for pesticidal compounds that are pest-specific, that reduce or eliminate direct and/or indirect threats to human health posed by presently available pesticides, that are environmentally compatible in the sense that they are biodegradable, are not toxic to non-pest organisms, and have reduced or no tendency to bioaccummulate.

Many pests, including for example blood-imbibing pests, must consume and digest a proteinaceous meal to acquire sufficient essential amino acids for growth, development and the production of mature eggs. Adult pests, such as adult mosquitoes, need these essential amino acids for the production of vitellogenins by the fat body. These vitellogenins are precursors to yolk proteins which are critical components of oogenesis. Many pests, such as house flies and mosquitoes, produce oostatic hormones that inhibit egg development by inhibiting digestion of the protein meal and thereby limiting the availability of the essential amino acids necessary for egg development.

Serine esterases such as trypsin and trypsin-like enzymes (collectively referred to herein as “TTLE”) are important components of the digestion of proteins by insects. In the mosquito, Aedes aegypti, an early trypsin that is found in the midgut of newly emerged females is replaced, following the blood meal, by a late trypsin. A female mosquito typically weighs about 2 mg and produces 4 to 6 ug of trypsin within several hours after ingesting a blood meal. Continuous boisynthesis at this rate would exhaust the available metabolic energy of a female mosquito; as a result, the mosquito would be unable to produce mature eggs, or even to find an oviposition site. To conserve metabolic energy, the mosquito regulates TTLE biosynthesis with a peptide hormone named Trypsin Modulating Oostatic Factor (TMOF). Mosquitoes produce TMOF in the follicular epithelium of the ovary 12-35 hours after a blood meal; TMOF is then released into the hemolymph where it binds to a specific receptor on the midgut epithelial cells, signaling the termination of TTLE biosynthesis. This regulatory mechanism is not unique for mosquitoes; flesh flies, fleas, sand flies, house flies, dog flies and other insect pests which need protein as part of their diet have similar regulatory mechanisms.

In 1985, Borovsky purified an oostatic hormone 7,000-fold and disclosed that injection of a hormone preparation into the body cavity of blood imbibed mosquitoes caused inhibition of egg development and sterility (Borovsky, D. [1985] Arch. Insect Biochem. Physiol. 2:333-349). Following these observations, Borovsky (Borovsky, D. [1988] Arch. Ins. Biochem. Physiol. 7:187-210) reported that injection or passage of a peptide hormone preparation into mosquitoes inhibited the TTLE biosynthesis in the epithelial cells of the gut. This inhibition caused inefficient digestion of the blood meal and a reduction in the availability of essential amino acids translocated by the hemolymph, resulting in arrested egg development in the treated insect. Borovsky observed that this inhibition of egg development does not occur when the oostatic hormone peptides are inside the lumen of the gut or other parts of the digestive system (Borovsky, D. [1988], supra).

Following the 1985 report, the isolated hormone, (a ten amino acid peptide) and two TMOF analogues were disclosed in U.S. Pat. Nos. 5,011,909 and 5,130,253, and in a 1990 publication (Borovsky et al. [1990] FASEB J. 4:3015-3020). Additionally, U.S. Pat. No. 5,358,934 discloses truncated forms of the full length TMOF which have prolines removed from the carboxy terminus, including the peptides YDPAP, YDPAPP, YDPAPPP, and YDPAPPPP.

D. Borovsky and R. Linderman, U.S. patent application Ser. No. 09/295,996, filed Apr. 21, 1999, discloses additional novel peptides and the use thereof to control insect pests.

TMOF analogs that have been identified to date are primarily peptide analogs. In order to provide a greater diversity of new pesticidal compounds, it would be desirable to possess compounds that are TMOF analogues, yet are not peptides.

### SUMMARY OF THE INVENTION

Thus, a first aspect of the present invention is a method of controlling a pest such as an insect pest, comprising administering to said pest a pesticidally effective amount of a non-peptide TMOF analog (also referred to as an “active compound” or “pesticidal compound” herein). Particular pesticidal compounds/non-peptide TMOF analogs of the present invention have the Formula IA or Formula IB below, or have the Formula IIA or Formula IIB below.

Thus, a first group of compounds of the present invention are compounds of Formula IA and Formula IB below:

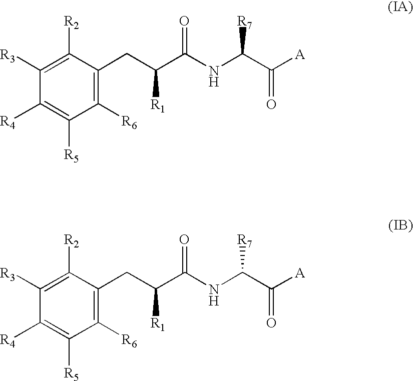

wherein:

R₁ is —H, —NH₂, or —OH;

R₂, R₃, R₄, R₅, and R₆ are each independently selected from the group consisting of H, halogen, hydroxyl, alkyl, alkylhydroxy, alkoxy, or phenyl;

or a pair of R₂ and R₃, R₃ and R₄, R₄ and R₅, and R₅ and R₆ together are —(CH)₄— to form a naphthyl group;

R₇ is H, alkyl, phenyl, alkylphenyl, or alkylcarboxy; and

A is selected from the group consisting of:

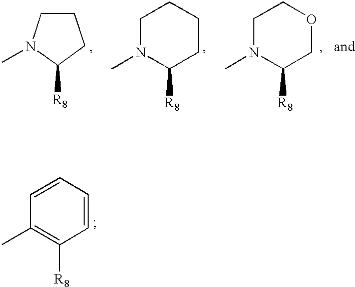

wherein R₈ is H, alkylhydroxy, or carboxy;

subject to the proviso that at least one of R₇ and R₈ is carboxy or alkylcarboxy;

and subject to the proviso that when R₁ is —NH₂, then one of R or R₈ is not carboxy or alkylcarboxy.

Additional compounds of the present invention are compounds of Formula IIA and Formula IIB below:

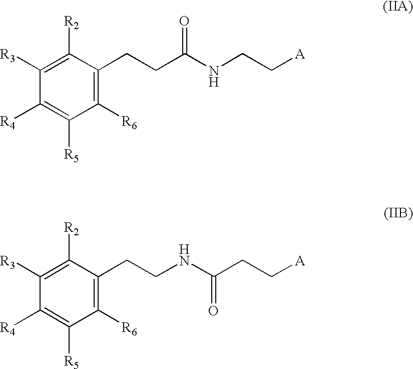

wherein:

R₂, R₃, R₄, R₅, and R₆ are each independently selected from the group consisting of H, halogen, hydroxyl, alkyl, alkylhydroxy, alkoxy, or phenyl;

or a pair of R₂ and R₃, R₃ and R₄, R₄ and R₅, and R₅ and R₆ together are —(CH)₄— to form a naphthyl group; and

A is selected from the group consisting of carboxy;

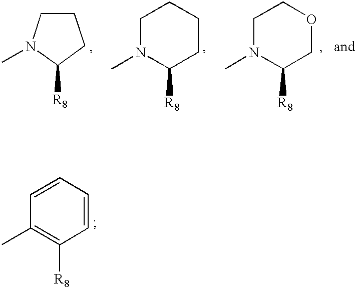

wherein R₈ is carboxy or alkylcarboxy.

A second aspect of the present invention is a method of initiating a TMOF receptor-mediated biological response. The method comprises contacting to a TMOF receptor in vivo or in vitro for a time and in an amount sufficient to initiate a TMOF receptor-mediated biological response a pesticidal compound as described herein. The biological response may be any suitable biological response mediated by the TMOF receptor, including but not limited to inhibition of biosynthesis of a digestive enzyme such as trypsin.

As noted above, the pesticidal compounds of the present invention have advantageous biological activity against pests. The novel compounds of the invention are particularly active against blood-sucking insects, particularly against species of mosquitoes such as Aedes aegypti that are common vectors of arthropod-borne viral diseases, such as arboviruses. Other biting pests such as flies, fleas, ticks, and lice can also be controlled using compounds and methods of the subject invention. These pests utilize TTLE as their primary blood-digesting enzymes.

The subject compounds can also be used to control pests of agricultural crops, for example by applying the compounds to the agricultural crops. These pests include, for example, coleopterans (beetles), lepidopterans (caterpillars), and mites. The compounds of the subject invention can also be used to control household pests including, but not limited to, ants and cockroaches.

Another aspect of the subject invention pertains to a method for controlling pests, particularly insect pests, comprising administering to said pest a pesticidally effective amount of a pesticidal compound of the subject invention.

The subject invention provides pest control compositions comprising pesticidal compounds and a suitable pesticidal carrier. The pest control compositions are formulated for application to the target pests or their situs.

The foregoing and other objects and aspects of the present invention are explained in greater detail in the drawings herein and the specification set forth below.

### DETAILED DESCRIPTION OF THE PREFERRED EMBODIMENTS

As used herein, the term “pesticidally effective” is used to indicate an amount or concentration of a pesticidal compound which is sufficient to reduce the number of pests in a geographical locus as compared to a corresponding geographical locus in the absence of the amount or concentration of the pesticidal compound.

The term “pesticidal” is not intended to refer only to the ability to kill pests, such as insect pests, but also includes the ability to interfere with a pest's life cycle in any way that results in an overall reduction in the pest population. For example, the term “pesticidal” includes inhibition of a pest from progressing from one form to a more mature form, e.g., transition between various larval instars or transition from larva to pupa or pupa to adult. Further, the term “pesticidal” is intended to encompass anti-pest activity during all phases of a pest's life cycle; thus, for example, the term includes larvacidal, ovicidal, and adulticidal activity.

As used herein, the term “alkyl” (e.g., alkyl, alkylxcarboxy, alkylphenyl, etc.) refers to a straight or branched chain hydrocarbon having from one to twelve carbon atoms, optionally substituted with substituents selected from the group which includes lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by a substituent selected from the group including alkyl, nitro, cyano, halogen and lower perfluoroalkyl, multiple degrees of substitution being allowed. Examples of “alkyl” as used herein include, but are not limited to, n-butyl, n-pentyl, isobutyl, isopropyl and the like. Loweralkyl is preferred.

The term “loweralkyl” as used herein means linear or branched C₁ to C₄ alkyl, preferably methyl, ethyl or propyl.

The term “loweralkoxy” as used herein means linear or branched C₁ to C₄ alkoxy, preferably methoxy, ethoxy, or propoxy.

The term “halo” as used herein means halogen, preferably fluoro, chloro, bromo or iodo, most preferably fluoro.

Certain of the compounds as described contain one or more chiral, or asymmetric, centers and are therefore be capable of existing as optical isomers that are either dextrorotatory or levorotatory. The invention includes the respective dextrorotatory or levorotatory pure preparations, as well as mixtures (racemic or enantiomerically enriched mixtures) thereof.

1. Pesticidal Compounds.

A first group of compounds of the present invention are compounds of Formula IA and IB below:

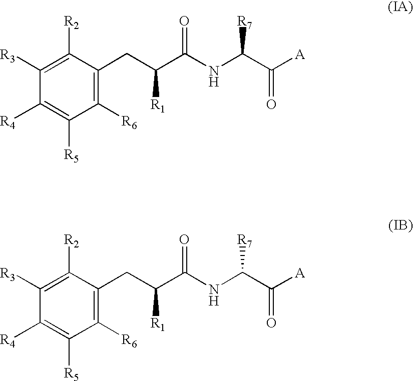

wherein:

R₁ is —H, —NH₂, or —OH;

R₂, R₃, R₄, R₅, and R₆ are each independently selected from the group consisting of H, halogen, hydroxyl, alkyl, alkylhydroxy, alkoxy, or phenyl;

or a pair of R₂ and R₃, R₃ and R₄, R₄ and R₅, and R₅ and R₆ together are —(CH)₄— to form a naphthyl group;

R₇ is H, alkyl, phenyl, alkylphenyl, or alkylcarboxy; and

A is selected from the group consisting of:

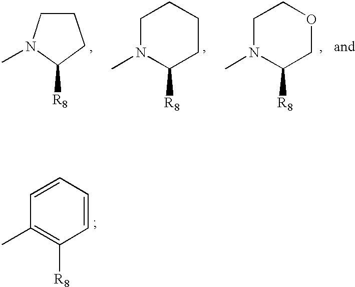

wherein R₈ is H, alkylhydroxy, or carboxy;

subject to the proviso that at least one of R₇ and R₈ is carboxy or alkylcarboxy; and

subject to the proviso that when R₁ is —NH₂, then one of R or R₈ is not carboxy or alkylcarboxy.

Specific examples of such compounds are compounds as set forth below:

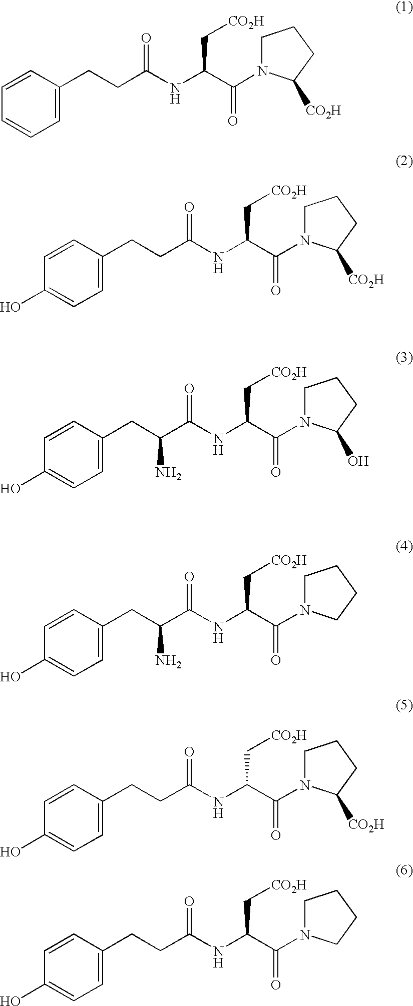

Additional compounds of the present invention are compounds of Formula IIA and IIB below:

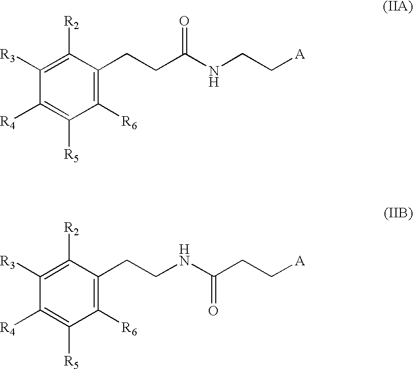

wherein:

R₂, R₃, R₄, R₅, and R₆ are each independently selected from the group consisting of H, halogen, hydroxyl, alkyl, alkylhydroxy, alkoxy, or phenyl;

or a pair of R₂ and R₃, R₃ and R₄, R₄ and R₅, and R₅ and R₆ together are —(CH)₄— to form a naphthyl group; and

A is selected from the group consisting of carboxy;

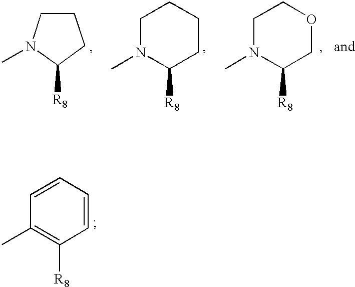

wherein R₈ is carboxy or alkylcarboxy.

Specific Examples of compounds of Formula IIA and IIB include, but are not limited to, the following:

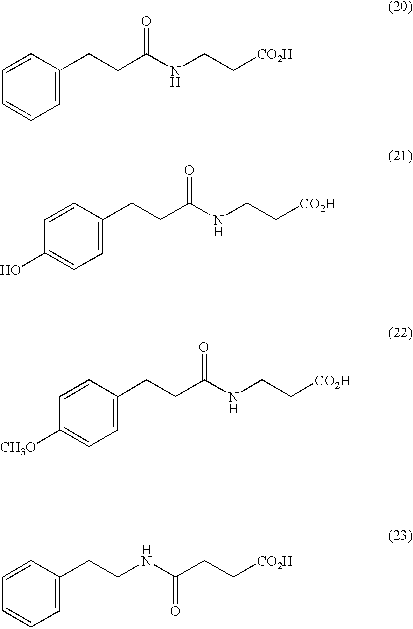

Compounds as described herein may be prepared by techniques known to those skilled in the art taken together with the information provided in the Examples set forth herein.

A further aspect of the subject invention are addition salts, complexes, or prodrugs such as esters of the compounds described herein, especially the nontoxic pharmaceutically or agriculturally acceptable acid addition salts. The acid addition salts can be prepared using standard procedures in a suitable solvent from the parent compound and an excess of an acid, such as hydrochloric, hydrobromic, sulfuric, phosphoric, acetic, maleic, succinic, ethanedisulfonic or methanesulfonic acids. Esterification to form derivatives such as the methyl or ethyl esters, can also be performed using standard procedures. Tartarate salts can be prepared in accordance with standard procedures.

Also, derivation of the pesticidal compounds with long chain hydrocarbons will facilitate passage through the cuticle into the pest body cavity. Therefore, in a further embodiment, the subject invention provides compositions comprising the pesticidal compounds bound to lipids or other carriers.

2. Methods and Formulations for Control of Pests.

The subject invention concerns novel pest control compounds and methods for using such compounds. Specifically exemplified are novel pesticidal compounds, compositions comprising said pesticidal compounds and the use of such pesticidal compounds and compositions in controlling pests, particularly insect pests such as mosquitoes Preferably, the subject compounds have an LD₅₀ against mosquito larvae of less than 3.0 μmole/ml. More preferably, the compounds have an LD₅₀ of less than 2.0 μmole/ml, and, most preferably, the compounds have an LD₅₀ of less than 1.0 μmole/ml. As used herein, “LD₅₀” refers to a lethal dose of a peptide able to cause 50% mortality of larvae maintained on a diet of 1 mg/ml autoclaved yeast supplemented with the pesticidal polypeptide.

Control of pests using the pest control compounds of the subject invention can be accomplished by a variety of methods known to those skilled in the art. The plant pests that can be controlled by the compounds of the subject invention include pests belonging to the orders Coleoptera, Lepidopterans, Hemiptera and Thysanoptera. These pests all belong to the phylum Arthropod. Other pests that can be controlled according to the subject invention include members of the orders Diptera, Siphonaptera, Hymenoptera and Phthiraptera. Other pests that can be controlled by the compounds of the subject invention include those in the family Arachnida, such as ticks, mites and spiders.

The use of the compounds of the subject invention to control pests can be accomplished readily by those skilled in the art having the benefit of the instant disclosure. For example, the compounds may be encapsulated, incorporated in a granular form, solubilized in water or other appropriate solvent, powdered, and included into any appropriate formulation for direct application to the pest or to a pest inhabited locus.

Formulated bait granules containing an attractant and the pesticidal compounds of the present invention can be applied to a pest-inhabited locus, such as to the soil. Formulated product can also be applied as a seed-coating or root treatment or total plant treatment at later stages of the crop cycle. Plant and soil treatments may be employed as wettable powders, granules or dusts, by mixing with various inert materials, such as inorganic minerals (phyllosilicates, carbonates, sulfates, phosphates, and the like) or botanical materials (powdered corncobs, rice hulls, walnut shells, and the like). The formulations may include spreader-sticker adjuvants, stabilizing agents, other pesticidal additives, or surfactants.

Liquid formulations may be aqueous-based or non-aqueous (i.e., organic solvents), or combinations thereof, and may be employed as foams, gels, suspensions, emulsions, microemulsions or emulsifiable concentrates or the like. The ingredients may include rheological agents, surfactants, emulsifiers, dispersants or polymers.

As would be appreciated by a person skilled in the art, the pesticidal concentration will vary widely depending upon the nature of the particular formulation, particularly whether it is a concentrate or to be used directly. The pesticidal compound will be present in the composition by at least about 0.0001% by weight and may be 99 or 100% by weight of the total composition. The pesticidal carrier may be from 0.1% to 99.9999% by weight of the total composition. The dry formulations will have from about 0.0001-95% by weight of the pesticide while the liquid formulations will generally be from about 0.0001-60% by weight of the solids in the liquid phase. These formulations will be administered at about 50 mg (liquid or dry) to 1 kg or more per hectare.

The formulations can be applied to the pest or the environment of the pest, e.g., soil and foliage, by spraying, dusting, sprinkling or the like.

The pest control compounds may also be provided in tablets, pellets, briquettes, bricks, blocks and the like which are formulated to float, maintain a specified depth or sink as desired. In one embodiment the formulations, according to the present invention, are formulated to float on the surface of an aqueous medium; in another embodiment they are formulated to maintain a depth of 0 to 2 feet in an aqueous medium; in yet another embodiment the formulations are formulated to sink in an aqueous environment.

The pesticidal compounds of the present invention may be used advantageously to control an insect population of a specific geographical area. The specific geographical area can be as large as a state or a county and is preferably ½ to 10 square miles, more preferably one square mile, and more preferably ½ to one square miles, and may also be much smaller, such as 100-200 square yards, or may simply include the environment surrounding and/or inside an ordinary building, such as a barn or house.

In general, the pesticidal compounds or compositions containing one or more of the pesticidal compounds are introduced to an area of infestation. For example, the composition can be sprayed on as a wet or dry composition on the surface of organic material infested with a target pest, or organic material or habitat susceptible to infestation with a target pest. Alternately, the composition can be applied wet or dry to an area of infestation where it can come into contact with the target pest. The pesticidal compound may also be applied to an area of larvae development, for example, an agricultural area or a body of water such as a pond, rice paddy, watering hole or even a small puddle.

In one aspect of the invention, a target pest population is exposed to a pesticidally effective amount of a pesticidal compound to decrease or eliminate the population of that pest in an area. The method of introduction of the pesticidal compound into the target pest can be by direct ingestion by the target pest from a trap, or by feeding of a target pest on nutrient-providing organic matter treated with the pesticidal compound, (e.g., killed yeast or algae in the case of mosquito larvae). For some applications it will be advantageous to deliver the pesticidal composition to the location of the pest colony. In other applications, it will be preferable to apply the pesticidal composition to a prey or host of the pest, such as a human or other animal.

Amounts and locations for application of the pesticidal compounds and compositions of the present invention are generally determined by the habits of the insect pest, the lifecycle stage at which the pest is to be attacked, the site where the application is to be made and the physical and functional characteristics of the compound.

The pesticidal compounds of the present invention are generally administered to the insect by oral ingestion, but may also be administered by means which permit penetration through the cuticle or penetration of the insect respiratory system. The pesticide may be absorbed by the pest, particularly where the composition provides for uptake by the outer tissues of the pest, particularly a larval or other pre-adult form of the pest, such as a detergent composition.

Where the pesticidal compounds are formulated to be orally administered to the insect pests, the compounds can be administered alone or in association with an insect food. The compounds are preferably so associated with the food that it is not possible for the insect to feed on the food without ingesting the pesticidal compound. Preferred foods for mosquito larvae are algae (particularly green, unicellular) and yeast. The food may comprise live organisms or killed organisms. In one embodiment for the control of plant pests, plants or other food organisms may be genetically transformed to express the pesticidal compound such that a pest feeding upon the plant or other food organism will ingest the pesticidal compound and thereby be controlled. The pesticidal compound may also be mixed with an attractant to form a bait that will be sought out by the pest. Further, the pesticidal compound may be applied as a systemic poison that is absorbed and distributed through the tissues of a plant or animal host, such that an insect feeding thereon will obtain an insecticidally effective dose of the pesticidal compound.

The compounds according to the present invention may be employed alone or in mixtures with one another and/or with such solid and/or liquid dispersible carrier vehicles as described herein or as otherwise known in the art, and/or with other known compatible active agents, including, for example, insecticides, acaricides, rodenticides, fungicides, bactericides, nematocides, herbicides, fertilizers, growth-regulating agents, etc., if desired, in the form of particular dosage preparations for specific application made therefrom, such as solutions, emulsions, suspensions, powders, pastes, and granules as described herein or as otherwise known in the art which are thus ready for use. For example, a dosage form for a pond environment may be provided in the form of time releasable bricks, briquettes, pellets, powders, liquids, and the like, comprising at least one pesticidal compound according to the present invention and at least one other active ingredient selected from the group consisting of insecticides, acaricides, rodenticides, fungicides, bactericides, nematocides, herbicides, fertilizers, and growth-regulating agents, for administration to the pond.

The pesticidal compounds may be administered with other insect control chemicals, for example, the compositions of the invention may employ various chemicals designed to affect insect behavior, such as attractants and/or repellents, or as otherwise known in the art. The pesticidal compounds may also be administered with chemosterilants.

The pesticidal compounds are suitably applied by any method known in the art including, for example, spraying, pouring, dipping, in the form of concentrated liquids, solutions, suspensions, sprays, powders, pellets, briquettes, bricks and the like, formulated to deliver a pesticidally effective concentration of the pesticidal compound. The pesticidal formulations may be applied in a pesticidally effective amount to an area of pest infestation or an area susceptible to infestation, a body of water or container, a barn, a carpet, pet bedding, an animal, clothing, skin, and the like.

Formulated pesticidal compounds can also be applied as a seed-coating or root treatment or total plant treatment at later stages of the crop cycle.

Plant and soil treatments may be employed as wettable powders, granules or dusts, by mixing with various inert materials, such as inorganic minerals (phyllosilicates, carbonates, sulfates, phosphates, and the like) or botanical materials (powdered corncobs, rice hulls, walnut shells, and the like). Such formulations suitably include spreader-sticker adjuvants, stabilizing agents, other pesticidal additives, or surfactants.

Liquid formulations may be aqueous-based or non-aqueous and employed as foams, gels, suspensions, emulsifiable concentrates, or the like.

The pesticidal compounds and compositions of the present invention can be delivered to the environment using a variety of devices known in the art of pesticide administration; particularly preferred devices are those which permit continuous extended or pulsed extended delivery of the pesticidal composition. For example, U.S. Pat. No. 5,417,682 discloses a fluid-imbibing dispensing device for the immediate, or almost immediate, and extended delivery of an active agent over a prolonged period of time together with the initially delayed pulse delivery of an active agent to a fluid environment of use.

Other dispensing means useful for dispensing the pesticidal compositions of the present invention include, for example, osmotic dispensing devices which employ an expansion means to deliver an agent to an environment of use over a period of hours, weeks, days or months. The expansion means absorbs liquid, expands, and acts to drive out beneficial agent composition from the interior of the device in a controlled, usually constant manner. An osmotic expansion device can be used to controllably, usually relatively slowly and over a period of time, deliver the pesticidal compositions of the present invention. The osmotic expansion device may be designed to float on water and deliver the pesticidal compound to the surface of the water.

The compositions of the present invention may also be employed as time-release compositions, particularly for applications to animals, or areas that are subject to reinfestation, such as mosquito-infested ponds or animal quarters. Various time-release formulations are known in the art. Common analytical chemical techniques are used to determine and optimize the rate of release to ensure the delivery of a pesticidally effective concentration of the pesticidal compound. The amount of the time-release composition necessary to achieve a pesticidally effective concentration of pesticide in the environment where the pesticide is applied, e.g., a body of water, is based on the rate of release of the time-release formulation. In one aspect, the time-release formulations may be formulated to float on top of the water. In another aspect, the formulation may be formulated to rest on the bottom, or below the surface of the body of water, and to gradually release small particles which themselves float to the surface, thereby delivering the pesticidal composition to the niche of the pest, e.g., mosquito larvae.

Delayed or continuous release can also be accomplished by coating the pesticidal compounds or a composition containing the pesticidal compound(s) with a dissolvable or bioerodable coating layer, such as gelatin, which coating dissolves or erodes in the environment of use, such as in a pond, to then make the pesticidal compound available, or by dispersing the compounds in a dissolvable or erodable matrix.

Such continuous release and/or dispensing means devices may be advantageously employed in a method of the present invention to consistently maintain a pesticidally effective concentration of one or more of the pesticidal compounds of the present invention in a specific pest habitat, such as a pond or other mosquito-producing body of water. The continuous release compositions are suitably formulated by means known in the art to float on a body of water, thereby delivering the pesticidal compound to the surface layer of the water inhabited by insect larvae.

The following examples are illustrative of the practice of the present invention and should not be construed as limiting. All percentages are by weight and all solvent mixture proportions are by volume unless otherwise noted.

### EXAMPLE 1

### Compound 1

The benzyl ester of (S)-proline hydrochloride was combined with the N-tert-butylcarbamate mono-benzyl ester of (S)-aspartic acid in methylene chloride. Peptide coupling was affected by the addition of dicyclohexylcarbodiimide, N-hydroxybenzotriazole, and N-ethylmorpholine at 0° C. and then allowing the mixture to warm to room temperature. The coupled dipeptide was then subjected to trifluoroacetic acid to remove the tert-butylcarbamate protecting group. The amine trifluoroacetate salt was then combined with 3-phenylpropionic acid, diisopropylethylamine, and BOP ((benzotriazol-1-yloxy)tris(dimethylamino)-phosphonium hexafluorophosphate) in methylene chloride at 0° C. The tripeptide derivative was obtained in 79% yield. The benzyl esters were then cleaved by hydrogenolysis (hydrogen at 1 atm using Pd/C as catalyst) in ethanol. The free diacid tripeptide, compound 1, was obtained in 85% yield. All new compounds were fully characterized by spectroscopic methods (infrared and nuclear magnetic resonance) and combustion analysis.

### EXAMPLE 2

### Compound 2

The benzyl ester of (S)-proline hydrochloride was combined with the N-tert-butylcarbamate mono-benzyl ester of (S)-aspartic acid in methylene chloride. Peptide coupling was affected by the addition of dicyclohexylcarbodiimide, N-hydroxybenzotriazole, and N-ethylmorpholine at 0° C., and then allowing the reaction mixture to warm to room temperature. The coupled dipeptide was then subjected to trifluoroacetic acid to remove the tert-butylcarbamate protecting group. The amine trifluoroacetate salt was then combined with 3-(4-hydroxyphenyl)propionic acid, dicyclohexylcarbodiimide, N-hydroxybenzotriazole, and N-ethylmorpholine at 0° C., and the reaction mixture was then allowed to warm to room temperature. The dipeptide amide dibenzyl ester was isolated in 60% yield. The benzyl esters were then cleaved by hydrogenolysis (hydrogen at 1 atm using Pd/C as catalyst) in ethanol. The free diacid dipeptide amide, compound 2, was obtained in 74% yield. All new compounds were fully characterized by spectroscopic methods (infrared and nuclear magnetic resonance) and combustion analysis.

### EXAMPLE 3

### Compound 3

The benzyl ether derivative of (S)-prolinol hydrochloride was combined with the N-tert-butylcarbamate mono-benzyl ester of (S)-aspartic acid in methylene chloride. Peptide coupling was affected by the addition of dicyclohexylcarbodiimide, N-hydroxybenzotriazole, and N-ethylmorpholine at 0° C., and then allowing the reaction mixture to warm to room temperature. The coupled dipeptide was then subjected to trifluoroacetic acid to remove the tert-butylcarbamate protecting group. The trifluoroacetate salt was exchanged to a p-toluenesulfonate salt, and the sulfonate salt was then coupled to the O-benzyl ether N-tert-butylcarbamate derivative of (S)-tyrosine using dicyclohexylcarbodiimide, N-hydroxybenzotriazole, and N-ethylmorpholine at 0° C. in tetrahydrofuran. The fully protected dipeptide amidederivative was obtained in 60% yield after chromatography. Deprotection of the tert-butylcarbamate was realized with trifluoroacetic acid, and the benzyl ether and benzyl esters were simultaneously removed by hydrogenolysis (hydrogen at 1 atm using Pd/C as catalyst) in ethanol. The unprotected dipeptide amide, compound 3, was obtained in 70% yield. All new compounds were fully characterized by spectroscopic methods (infrared and nuclear magnetic resonance) and combustion analysis.

### EXAMPLE 4

### Compound 4

Pyrrolidine was combined with the N-tert-butylcarbamate mono-benzyl ester of (S)-aspartic acid in methylene chloride. Amide coupling was affected by the addition of dicyclohexylcarbodiimide, N-hydroxybenzotriazole, and N-ethylmorpholine at 0° C., and then allowing the reaction mixture to warm to room temperature. The protected amino acid amide derivative was then subjected to trifluoroacetic acid to remove the tert-butylcarbamate protecting group. The trifluoroacetate salt was then coupled to the O-benzyl ether N-tert-butylcarbamate derivative of (S)-tyrosine using dicyclohexylcarbodiimide, N-hydroxybenzotriazole, and N-ethylmorpholine at 0 C. in tetrahydrofuran. The fully protected dipeptide amide derivative was obtained in 54% yield after chromatography. Deprotection of the tert-butylcarbamate was realized with trifluoroacetic acid and the benzyl ether and benzyl ester were simultaneously removed by hydrogenolysis (hydrogen at 1 atm using Pd/C as catalyst) in ethanol. The dipeptide amide, compound 4, was obtained in 60% yield. All new compounds were fully characterized by spectroscopic methods (infrared and nuclear magnetic resonance) and combustion analysis.

### EXAMPLE 5

### Compound 5

The benzyl ester of (S)-proline hydrochloride was combined with the N-tert-butylcarbamate mono-benzyl ester of (R)-aspartic acid in methylene chloride. Peptide coupling was affected by the addition of BOP ((benzotriazol-1-yloxy)tris(dimethylamino)-phosphonium hexafluorophosphate) and diisopropylethylamine at 0° C., and then allowing the reaction mixture to warm to room temperature. The coupled dipeptide was then subjected to trifluoroacetic acid to remove the tert-butylcarbamate protecting group. The amine trifluoroacetate salt was then combined with 3-(4-hydroxyphenyl)propionic acid, diisopropylethylamine and BOP ((benzotriazol-1-yloxy)tris(dimethylamino)-phosphonium hexafluorophosphate) in methylene chloride at 0° C. The dipeptide amide derivative was obtained in 73% overall yield. The benzyl esters were then cleaved by hydrogenolysis (hydrogen at 1 atm using Pd/C as catalyst) in ethanol. The free diacid dipeptide amide, compound 5, was obtained in 92% yield. All new compounds were fully characterized by spectroscopic methods (infrared and nuclear magnetic resonance) and combustion analysis.

### EXAMPLE 6

### Compound 6

Pyrrolidine was combined with the N-tert-butylcarbamate mono-benzyl ester of (S)-aspartic acid in methylene chloride. Amide coupling was affected by the addition of dicyclohexylcarbodiimide, N-hydroxybenzotriazole, and N-ethylmorpholine at 0° C., and then allowing the reaction mixture to warm to room temperature. The protected amino acid amide derivative was then subjected to trifluoroacetic acid to remove the tert-butylcarbamate protecting group. The trifluoroacetate salt was then coupled to 3-(4-hydroxyphenyl)propionic acid using diisopropylethylamine and BOP ((benzotriazol-1-yloxy)tris(dimethylamino)-phosphonium hexafluorophosphate) in methylene chloride at 0° C. The benzyl ester was then cleaved by hydrogenolysis (hydrogen at 1 atm using Pd/C as catalyst) in ethanol. The free acid dipeptide amide, compound 6, was obtained in 96% yield. All new compounds were fully characterized by spectroscopic methods (infrared and nuclear magnetic resonance) and combustion analysis.

### EXAMPLE 7

### Compound 20

Dihydrocinnamic acid was combined with the hydrochloride salt of ethyl 3-aminopropionate in the presence of dicyclohexylcarbodiimide, triethylamine, and a catalytic amount of 4-N,N-dimethylaminopyridine in methylene chloride. The ester-amide intermediate was purified by chromatography and then subjected to saponification using sodium hydroxide in methanol/water at room temperature. The acid, compound 20, was obtained in 77% yield. All new compounds were fully characterized by spectroscopic methods (infrared and nuclear magnetic resonance) and combustion analysis.

### EXAMPLE 8

### Compound 21

Compound 21 was prepared in substantially the same fashion as compound 20 by substitution of 3-(4-hydroxyphenyl)propionic acid for dihydrocinnamic acid in the first step of the sequence. All new compounds were fully characterized by spectroscopic methods (infrared and nuclear magnetic resonance) and combustion analysis.

### EXAMPLE 9

### Compound 22

Compound 22 was prepared in substantially the same fashion as compound 20 by substitution of 3-(4-methoxyphenyl)propionic acid for dihydrocinnamic acid in the first step of the sequence. All new compounds were fully characterized by spectroscopic methods (infrared and nuclear magnetic resonance) and combustion analysis.

### EXAMPLE 10

### Compound 23

2-Phenylethyl amine was combined with succinic anhydride and triethylamine in tetrahydrofuran at room temperature. The reaction mixture was then subjected to an aqueous work-up and acidified to pH 3 using dilute aqueous hydrochloric acid. Compound 23 was isolated in 65% yield. All new compounds were fully characterized by spectroscopic methods (infrared and nuclear magnetic resonance) and combustion analysis.

The foregoing is illustrative of the present invention, and is not to be construed as limiting thereof. The invention is defined by the following claims, with equivalents of the claims to be included therein.

That which is claimed is:

1. A method for controlling a pest, comprising administering to said pest a pesticidally effective amount of a pesticidal compound of Formula IA or Formula IB: 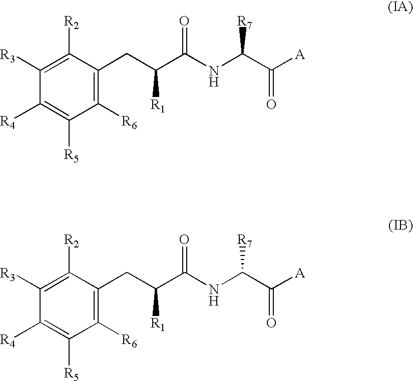
wherein:
R₁ is —H, —NH₂, or —OH;
R₂, R₃, R₄, R₅, and R₆ are each independently selected from the group consisting of H, halogen, hydroxyl, alkyl, alkylhydroxy, alkoxy, or phenyl;
or a pair of R₂ and R₃, R₃ and R₄, R₄ and R₅, and R₅ and R₆ together are —(CH)₄— to form a naphthyl group;
R₇ is H, alkyl, phenyl, alkylphenyl, or alkylcarboxy; and
A is selected from the group consisting of: 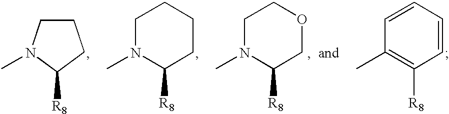
wherein R₈ is H, alkylhydroxy, or carboxy;
subject to the proviso that at least one of R₇ and R₈ is carboxy or alkylcarboxy;
and subject to the proviso that when R₁ is —NH₂, then one of R₁ or R₈ is not carboxy or alkylcarboxy.

2. A method according to claim 1, wherein said pest is an insect pest.

3. A method according to claim 1, wherein said pest is an insect selected from the group consisting of coleopterans, lepidopterans, and dipterans.

4. A method according to claim 1, wherein said pest is a blood-sucking insect.

5. A method according to claim 1, wherein said pest is an insect of the suborder Nematocera.

6. A method according to claim 1, wherein said pest is an insect of the family Colicidae.

7. A method according to claim 1, wherein said pest is an insect of a subfamily selected from the group consisting of Culicinae, Corethrinae, Ceratopogonidae and Simuliidae.

8. A method according to claim 1, wherein said pest is an insect of a genus selected from the group consisting of Culex, Theobaldia, Aedes, Anopheles, Aedes, Forciponiyia, Culicoides and Helea.

9. A method according to claim 1, wherein said pest is an insect species selected from the group consisting of: Aedes aegypti, Culex quinquefasciatus, Anopheles albimanus, Anopheles quadrimaculatus, Lutzomyia anthrophora, Culicoides variipennis, Stomoxys calcitrans, Musca domestica, Ctenocephalides feliz, and Heliothis virescens.

10. A method according to claim 1, wherein said pest is selected from the group consisting of flies, fleas, ticks, and lice.

11. A method according to claim 1, wherein said pest is a mosquito.

12. A method according to claim 1, wherein said pest is selected from the group consisting of beetles, caterpillars, and mites.

13. A method according to claim 1, wherein said pest is selected from the group consisting of ants and cockroaches.

14. A method according to claim 1, wherein said compound of Formula IA or Formula IB is selected from the group consisting of:

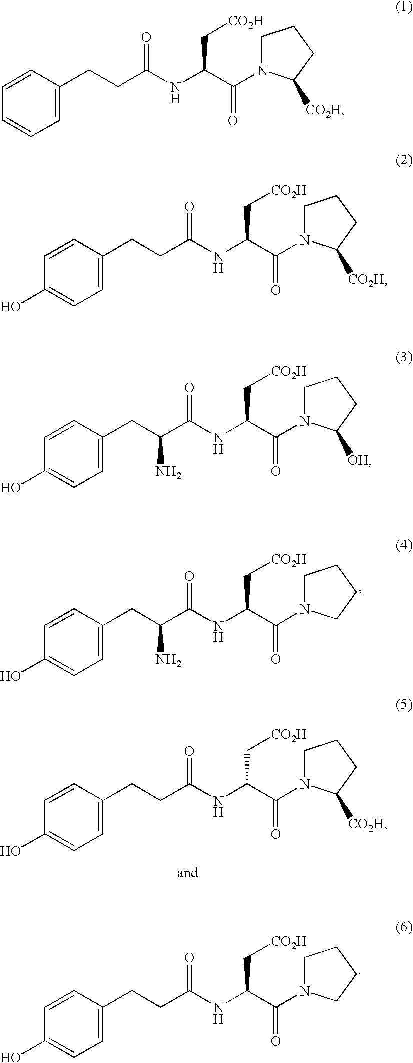

15. A method of initiating a TMOF receptor-mediated biological response, comprising contacting to a TMOF receptor in vivo or in vitro for a time and in an amount sufficient to initiate a TMOF receptor-mediated biological response a compound of Formula IA or Formula IB: 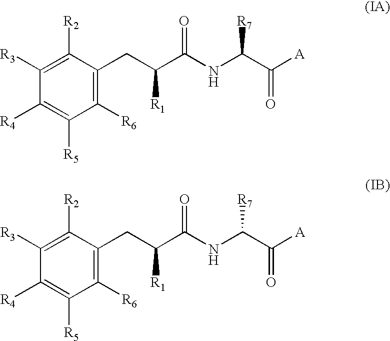
wherein:
R₁ is —H, —NH₂, or —OH;
R₂, R₃, R₄, R₅, and R₆ are each independently selected from the group consisting of H, halogen, hydroxyl, alkyl, alkylhydroxy, alkoxy, or phenyl;
or a pair of R₂ and R₃, R₃ and R₄, R₄ and R₅, and R₅ and R₆ together are —(CH)₄— to form a naphthyl group;
R₇ is H, alkyl, phenyl, alkylphenyl, or alkylcarboxy; and
A is selected from the group consisting of: 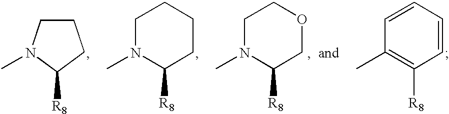
wherein R₈ is H, alkylhydroxy, or carboxy;
subject to the proviso that at least one of R₇ and R₈ is carboxy or alkylcarboxy;
and subject to the proviso that when R₁ is —NH₂, then one of R or R₈ is not carboxy or alkylcarboxy.

16. A method according to claim 15, wherein said biological response is inhibition of biosynthesis of a digestive enzyme.

17. A method according to claim 15, wherein said digestive enzyme is trypsin.

18. A method according to claim 15, wherein said contacting step is carried out in vivo in an insect pest.

19. A method according to claim 15, wherein said compound is selected from the group consisting of:

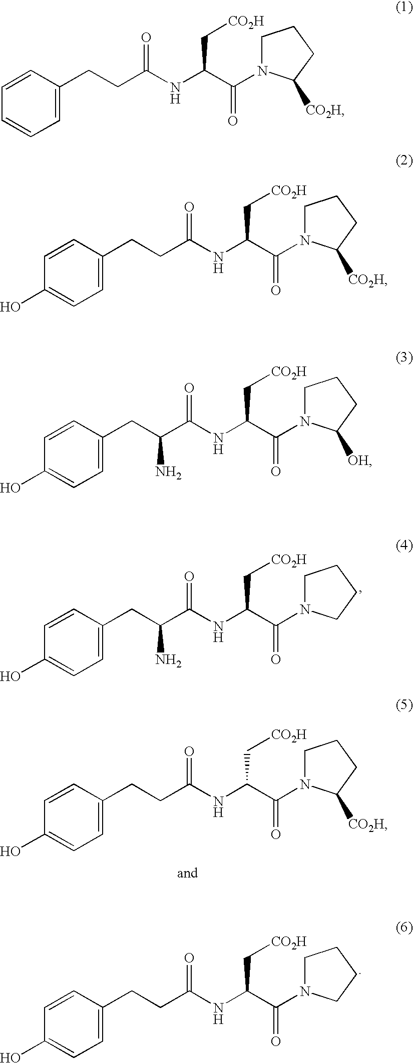

20. A pest control composition comprising a pesticidal carrier and a pesticidal compound of Formula IA or Formula IB: 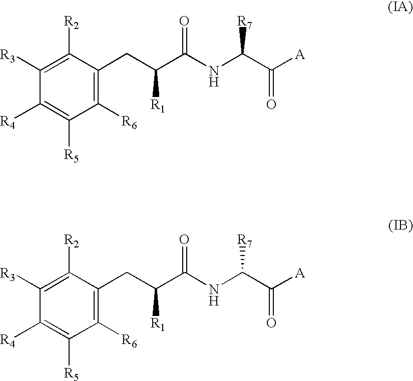
wherein:
R₁ is —H, —NH₂, or —OH;
R₂, R₃, R₄, R₅, and R₆ are each independently selected from the group consisting of H, halogen, hydroxyl, alkyl, alkylhydroxy, alkoxy, or phenyl;
or a pair of R₂ and R₃, R₃ and R₄, R₄ and R₅, and R₅ and R₆ together are —(CH)₄— to form a naphthyl group;
R₇ is H, alkyl, phenyl, alkylphenyl, or alkylcarboxy; and
A is selected from the group consisting of: 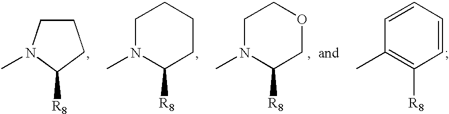
wherein R₈ is H, alkylhydroxy, or carboxy;
subject to the proviso that at least one of R₇ and R₈ is carboxy or alkylcarboxy;
and subject to the proviso that when R₁ is —NH₂, then one of R or R₈ is not carboxy or alkylcarboxy.

21. A composition according to claim 20, wherein said composition is a liquid composition.

22. A composition according to claim 20, wherein said pesticidal carrier is included in said composition in an amount from 0.1% to 99.9999% by weight.

23. A composition according to claim 20, wherein said pesticidal carrier comprises an aqueous solution.

24. A composition according to claim 20, wherein said pesticidal carrier comprises an organic solvent.

25. A composition according to claim 20, wherein said pesticidal carrier comprises an emulsion.

26. A composition according to claim 20, wherein said composition is a solid composition.

27. A composition according to claim 20, wherein said composition is a bait granule.

28. A composition according to claim 20, wherein said compound of Formula IA or Formula IB is selected from the group consisting of:

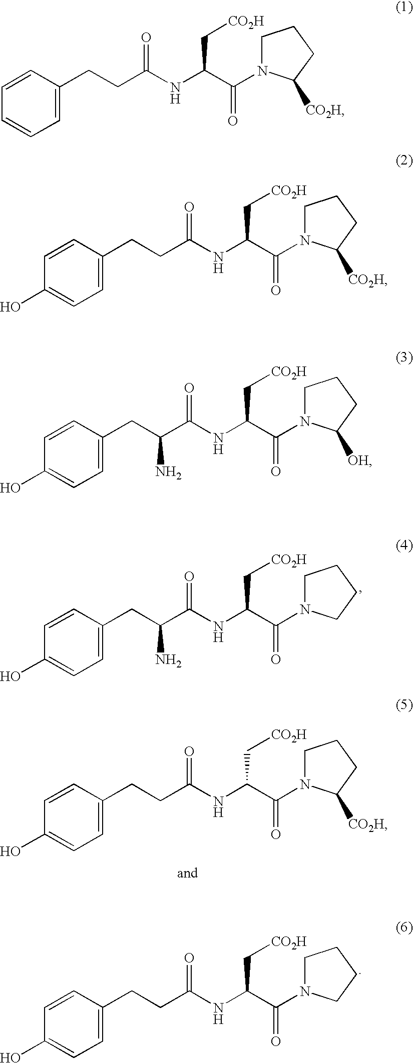

29. A compound of Formula IA or Formula IB: 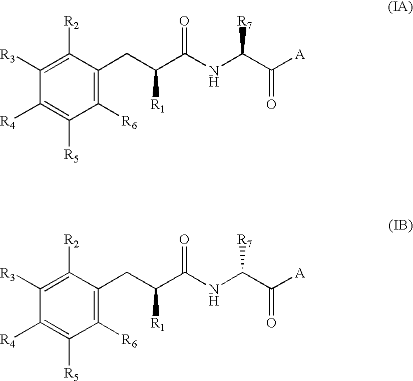
wherein:
R₁ is —H, —NH₂, or —OH;
R₂, R₃, R₄, R₅, and R₆ are each independently selected from the group consisting of H, halogen, hydroxyl, alkyl, alkylhydroxy, alkoxy, or phenyl;
or a pair of R₂ and R₃, R₃ and R₄, R₄ and R₅, and R₅ and R₆ together are —(CH)₄— to form a naphthyl group;
R₇ is H, alkyl, phenyl, alkylphenyl, or alkylcarboxy; and
A is selected from the group consisting of: 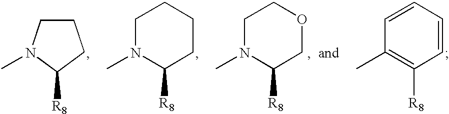
wherein R₈ is H, alkylhydroxy, or carboxy;
subject to the proviso that at least one of R₇ and R₈ is carboxy or alkylcarboxy;
and subject to the proviso that when R₁ is —NH₂, then one of R or R₈ is not carboxy or alkylcarboxy.

30. A compound according to claim 29, said compound selected from the group consisting of:

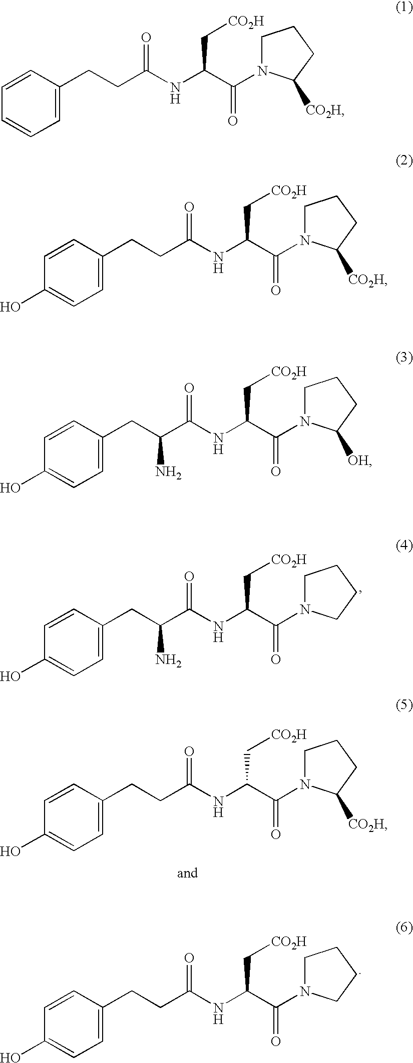

31. A method for controlling a pest, comprising administering to said pest a pesticidally effective amount of a pesticidal compound of Formula IIA or Formula IIB: 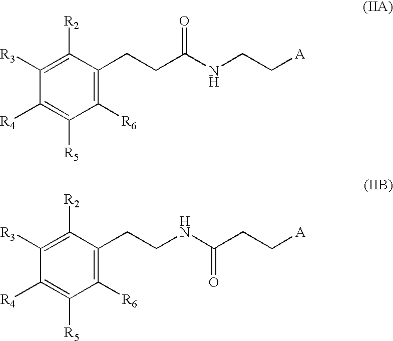
wherein:
R₂, R₃, R₄, R₅, and R₆ are each independently selected from the group consisting of H, halogen, hydroxyl, alkyl, alkylhydroxy, alkoxy, or phenyl;
or a pair of R₂ and R₃, R₃ and R₄, R₄ and R₅, and R₅ and R₆ together are —(CH)₄— to form a naphthyl group; and
A is selected from the group consisting of carboxy; 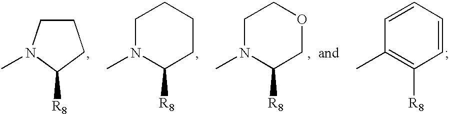
wherein R₈ is carboxy or alkylcarboxy.

32. A method according to claim 31, wherein said pest is an insect pest.

33. A method according to claim 31, wherein said pest is an insect selected from the group consisting of coleopterans, lepidopterans, and dipterans.

34. A method according to claim 31, wherein said pest is a blood-sucking insect.

35. A method according to claim 31, wherein said pest is an insect of the suborder Nematocera.

36. A method according to claim 31, wherein said pest is an insect of the family Colicidae.

37. A method according to claim 31, wherein said pest is an insect of a subfamily selected from the group consisting of Culicinae, Corethrinae, Ceratopogonidae and Simuliidae.

38. A method according to claim 31, wherein said pest is an insect of a genus selected from the group consisting of Culex, Theobaldia, Aedes, Anopheles, Aedes, Forciponiyia, Culicoides and Helea.

39. A method according to claim 31, wherein said pest is an insect species selected from the group consisting of: Aedes aegypti, Culex quinquefasciatus, Anopheles albimanus, Anopheles quadrimaculatus, Lutzomyia anthrophora, Culicoides variipennis, Stomoxys calcitrans, Musca domestica, Ctenocephalides feliz, and Heliothis virescens.

40. A method according to claim 31, wherein said pest is selected from the group consisting of flies, fleas, ticks, and lice.

41. A method according to claim 31, wherein said pest is a mosquito.

42. A method according to claim 31, wherein said pest is selected from the group consisting of beetles, caterpillars, and mites.

43. A method according to claim 31, wherein said pest is selected from the group consisting of ants and cockroaches.

44. A method according to claim 31, wherein said compound of Formula IIA or Formula IIB is selected from the group consisting of:

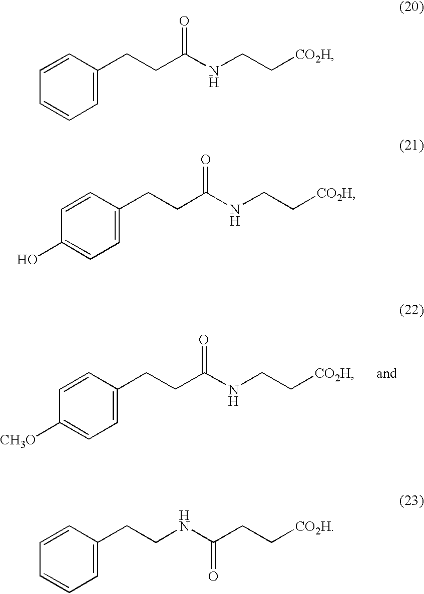

45. A method of initiating a TMOF receptor-mediated biological response, comprising contacting to a TMOF receptor in vivo or in vitro for a time and in an amount sufficient to initiate a TMOF receptor-mediated biological response a compound of Formula IIA or Formula IIB: 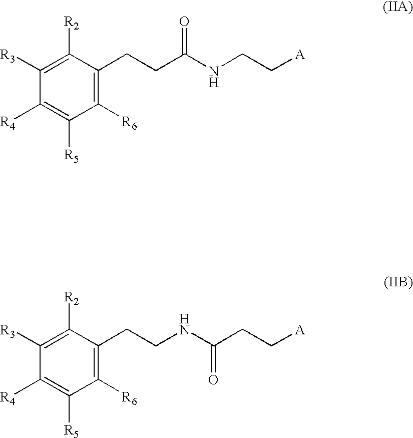
wherein:
R₂, R₃, R₄, R₅, and R₆ are each independently selected from the group consisting of H, halogen, hydroxyl, alkyl, alkylhydroxy, alkoxy, or phenyl;
or a pair of R₂ and R₃, R₃ and R₄, R₄ and R₅, and R₅ and R₆ together are —(CH)₄— to form a naphthyl group; and
A is selected from the group consisting of carboxy; 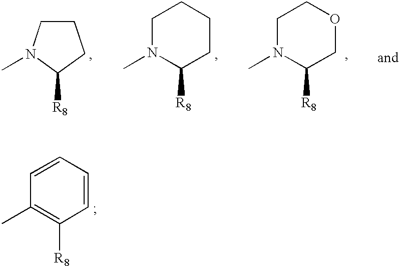
wherein R₈ is carboxy or alkylcarboxy.

46. A method according to claim 45, wherein said biological response is inhibition of biosynthesis of a digestive enzyme.

47. A method according to claim 45, wherein said digestive enzyme is trypsin.

48. A method according to claim 45, wherein said contacting step is carried out in vivo in an insect pest.

49. A method according to claim 45, wherein said compound is selected from the group consisting of:

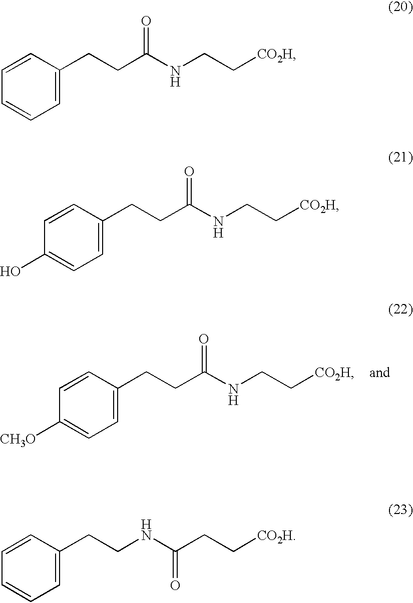

50. A pest control composition comprising a pesticidal carrier and a pesticidal compound of Formula IIA or Formula IIB: 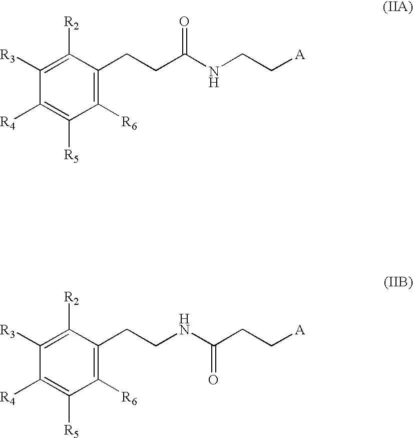
wherein:
R₂, R₃, R₄, R₅, and R₆ are each independently selected from the group consisting of H, halogen, hydroxyl, alkyl, alkylhydroxy, alkoxy, or phenyl;
or a pair of R₂ and R₃, R₃ and R₄, R₄ and R₅, and R₅ and R₆ together are —(CH)₄— to form a naphthyl group; and
A is selected from the group consisting of carboxy; 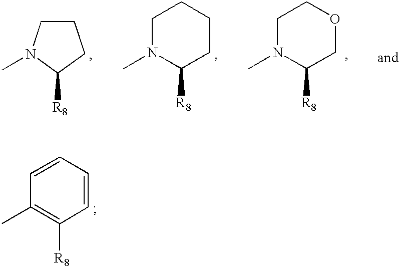
wherein R₈ is carboxy or alkylcarboxy.

51. A composition according to claim 50, wherein said composition is a liquid composition.

52. A composition according to claim 50, wherein said pesticidal carrier is included in said composition in an amount from 0.1% to 99.9999% by weight.

53. A composition according to claim 50, wherein said pesticidal carrier comprises an aqueous solution.

54. A composition according to claim 50, wherein said pesticidal carrier comprises an organic solvent.

55. A composition according to claim 50, wherein said pesticidal carrier comprises an emulsion.

56. A composition according to claim 50, wherein said composition is a solid composition.

57. A composition according to claim 50, wherein said composition is a bait granule.

58. A composition according to claim 50, wherein said compound of Formula IIA or Formula IIB is selected from the group consisting of:

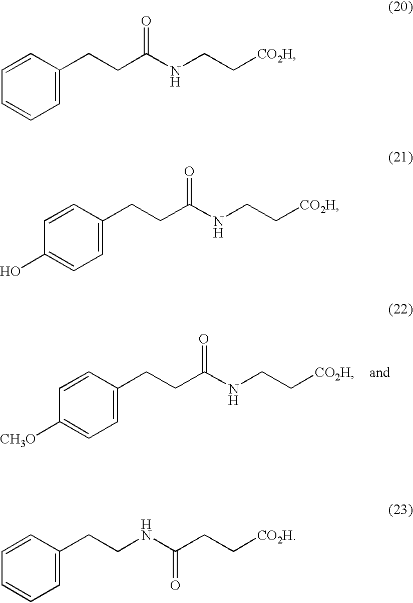

59. A compound of Formula IIA or Formula IIB: 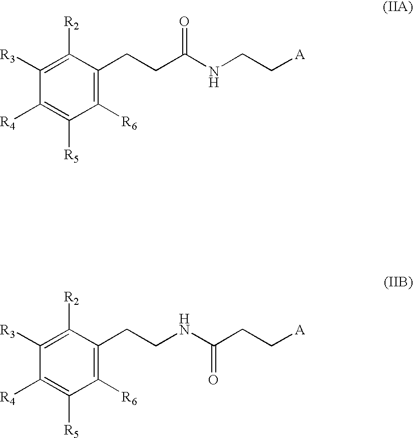
wherein:
R₂, R₃, R₄, R₅, and R₆ are each independently selected from the group consisting of H, halogen, hydroxyl, alkyl, alkylhydroxy, alkoxy, or phenyl;
or a pair of R₂ and R₃, R₃ and R₄, R₄ and R₅, and R₅ and R₆ together are —(CH)₄— to form a naphthyl group; and
A is selected from the group consisting of carboxy; 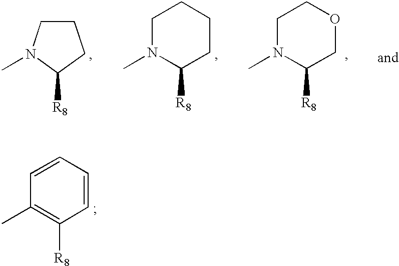
wherein R₈ is carboxy or alkylcarboxy.

60. A compound according to claim 59 selected from the group consisting of: [chemical structure]
